SEQUENCE LISTING (14 sequences)

SEQ ID NO: 1 - protein, 15 aa, Mus musculus
GGGYDFGYDGDFYRA

SEQ ID NO: 2 - protein, 116 aa, Mus musculus
QAQIQLVQSGPELKKPGETVKISCKASGYTFTDYPLHWVKQAPGKGLQWMAWINTETGEPTYADDFTGRFAFSLETSASTAYLQINNLKNEDTATYFCVRGYYYYWGQGTTLSVSS

SEQ ID NO: 3 - protein, 10 aa, Mus musculus
GYTFTDYPLH

SEQ ID NO: 4 - protein, 17 aa, Mus musculus
WINTETGEPTYADDFTG

SEQ ID NO: 5 - protein, 5 aa, Mus musculus
GYYYY

SEQ ID NO: 6 - protein, 113 aa, Mus musculus
DIVMSQSPSSLAVSAGEKVTMSCKSSQSLLNSRTRKNNLAWYQQKPGQSPKLLIYWASTRESGVPDRFTGSGSGTDFTLTISSVQAEDLAVYYCKQSYNLWTFGGGTKLEIKR

SEQ ID NO: 7 - protein, 17 aa, Mus musculus
KSSQSLLNSRTRKNNLA

SEQ ID NO: 8 - protein, 7 aa, Mus musculus
WASTRES

SEQ ID NO: 9 - protein, 8 aa, Mus musculus
KQSYNLWT

SEQ ID NO: 10 - protein, 115 aa, Mus musculus
QAQIQLVQSGPELKKPGETVKISCKTSGYTFTDYSMHWVKQAPGKGLKWMGWINTETGGPTYADDFKGRFAFSLETSASTAYLQINNLKNEDTATYFCARTSVYWGQGTTLTVSS

SEQ ID NO: 11 - protein, 120 aa, Mus musculus
AQIQLVQSGPELKKPGETVKISCKASGYTFTDYSMHWVKQAPGKGLKWMGWINTETGEPTYADDFKGRFAFSLETSASTAHLQINNLKNEDTATYFCARETAFFDDFTVWGQGTTLTVSS

SEQ ID NO: 12 - protein, 95 aa, Mus musculus
QAQIQLVQSGPELKKPGETVKISCKASGYTFTDYSMHWVKQAPGKGLKWMGWINTETGEPTYADDFKGRFAFSLETSASTAYLQINNLKNEDTAT

SEQ ID NO: 13 - protein, 113 aa, Mus musculus
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLWTFGGGTKLEIKR

SEQ ID NO: 14 - protein, 113 aa, Mus musculus
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLYTFGGGTKLEIKR

(19) United States
(12) Patent Application Publication
(10) Pub. No.: US 2020/0048333 A1
(43) Pub. Date: Feb. 13, 2020

(54) ANTIGEN-BINDING DOMAINS OF THE MONOCLONAL ANTI-COLLAGEN I ANTIBODY

(71) Applicant: THOMAS JEFFERSON UNIVERSITY, Philadelphia, PA (US)

(72) Inventors: Andrzej FERTALA, Voorhees, NJ (US); Andrzej STEPLEWSKI, Phoenixville, PA (US)

(21) Appl. No.: 16/345,587

(22) PCT Filed: Oct. 26, 2017
(86) PCT No.: PCT/US2017/058551

§ 371 Date: Apr. 26, 2019

Related U.S. Application Data
(60) Provisional application No. 62/413,235, filed on Oct. 26, 2016.

(51) Int. Cl. C07K 16/18

(52) U.S. Cl. CPC C07K 16/18; C07K 2317/565; C07K 2317/92; A61K 45/06; C07K 2317/24; C07K 2317/41; C07K 2317/622; C07K 2317/34

(57) ABSTRACT

An anti-fibrotic biologic comprising, a full-length chimeric IgG variant, a humanized IgG variant, a scFv variant, or other active biologic including the entire CDRs or their fragments able to bind to the α2Ct target.

### CROSS-REFERENCE TO RELATED APPLICATIONS

The present application claims priority to U.S. Provisional Patent Application No. 62/413,235, filed Oct. 26, 2016, which is hereby incorporated by reference in its entirety.

### SEQUENCE LISTING

The instant application contains a Sequence Listing which has been submitted in ST. 25 Text File Format via EFS-WEB and is hereby incorporated by reference in its entirety.

### FIELD OF INVENTION

The present invention is generally related to antigen-binding domains of monoclonal antibodies having binding for Collagen I.

### BACKGROUND OF INVENTION

Collagen I is the most abundant structural protein of connective tissues such as skin, bone, and tendon. This protein is first synthesized as a precursor molecule, procollagen I, that is characterized by the presence of a rod-like central triple-helical domain flanked by short linear telopeptides and globular N-terminal and C-terminal propeptides (1). Single procollagen I molecules are the building blocks for the biologically and mechanically relevant collagen fibrils. Formation of collagen fibrils is initiated by enzymatic cleavage of the N-terminal and the C-terminal propeptides. The N-terminal propeptides are cleaved by a group of enzymes that includes a disintegrin and metalloprotease with thrombospondin motifs (ADAMTS)-2, -3, and -14, whereas the C-terminal propeptides are cleaved by the metalloprotease bone morphogenetic protein 1 (BMP-1) and by the other members of a closely related family of mammalian tolloid-like metalloproteases (2-4). Such a removal of procollagen propeptides exposes telopeptides, which by engaging in site-specific intermolecular interactions drive collagen self-assembly.

In native tissues a precise balance between the processes of biosynthesis and degradation maintains the physiological homeostasis of tissue collagens. At the same time, accelerated biosynthesis is required for proper wound healing, whereas excessive accumulation of collagen is the hallmark of a number of localized fibrotic diseases, such as keloids and hypertrophic scars, and systemic fibrosis, such as systemic scleroderma.

Localized fibrotic reactions are quite common and frequently develop as a consequence of surgical procedures. For instance, after surgery of the abdomen, the formation of excessive scar tissue around abdominal organs, such as the intestines, can interfere with the functionality of such organs and may cause severe pain and even death. Another situation where excessive scar formation presents a major complication is in the eye after glaucoma surgery performed to create a pressure maintenance valve. Frequently, however, excessive scar formation closes this pressure-reducing valve, thereby forcing the intraocular pressure to rise (5). Moreover, excessive scarring of the vocal folds may severely alter their ability to vibrate, thereby causing a number of voice disorders (6).

At present, several biological processes critical for development of fibrotic lesions are considered potential targets for inhibitors of fibrosis. These inhibitors aim at (i) reducing inflammatory processes associated with fibrosis, (ii) inhibiting biological functions of cytokines and growth factors that promote fibrosis, (iii) reducing cell proliferation, and (iv) decreasing biosynthesis and processing of procollagens. Because most of those potential targets are involved not only in pathological fibrosis but also in a number of physiological processes, their inhibition is frequently associated with significant adverse effects (7-11).

At present, therapeutic approaches to limit fibrotic response target broad intracellular processes associated with inflammation and cell proliferation. Consequently, these approaches are non-specific and frequently associated with unwanted side effects. In contrast, limiting the growth of fibrotic tissue by directly blocking the extracellular process of collagen fibril formation with the use of the anti-fibrotic antibody (AFA) described herein, offers a novel and highly-specific therapeutic approach.

### SUMMARY OF INVENTION

The invention presented here is the amino acid sequences of the complementarity determining regions (CDRs) of the heavy alpha chain and the light kappa chain of a monoclonal antibody (denoted as anti-fibrotic antibody, AFA) that blocks the binding activity of the C-terminal telopeptide region of human collagen I (denoted as CTTR1) consisting of two α1(I)C-telopeptides (denoted as α1Ct) and one α2(I)C-telopeptide (denoted as α2Ct). These CDRs mediate the blocking of the CTTR1 via binding to its specific subdomain. Specifically, these CDRs mediate the binding interaction with a domain that includes a unique epitope, (denoted as A2_DGDFY) present within the α2Ct, with a minimum binding affinity of 22 μM.

A preferred embodiment of this invention is to apply the CDRs-containing antibody-based biologics in systemic or localized fibrotic diseases to limit the progression of the fibrotic process.

A further preferred embodiment of this invention includes targeted delivery of therapeutic compounds to collagen I-rich connective tissues. We envision that a highly-specific binding mediated by the described CDRs-CTTR1 interaction may serve to deliver therapeutic agents including antibiotics, growth factors, therapeutic cells, and others. Our published data support this concept. The end product will be an anti-fibrotic biologic: specifically, a full-length chimeric IgG variant, a humanized IgG variant, an scFv variant, or other active biologic including the entire CDRs or their fragments able to bind to the α2Ct target.

A monoclonal antibody comprising the amino acid sequences of the complementarity determining regions (CDRs) of the heavy alpha chain and the light kappa chain of a monoclonal antibody (denoted as anti-fibrotic antibody, AFA) that blocks the binding activity of the C-terminal telopeptide region of human collagen I (denoted as CTTR1) consisting of two α1(I)C-telopeptides (denoted as α1Ct) and one α2(I)C-telopeptide (denoted as α2Ct). These CDRs mediate the blocking of the CTTR1 via binding to its specific subdomain.

In further embodiments, the monoclonal antibody as above, wherein the CDRs mediate the binding interaction with a specific region that includes an epitope, (denoted as A2_DGDFY) present within the α2Ct, with a minimum binding affinity of 22 μM. In further embodiments, the monoclonal antibody having the sequence according to SEQ ID No 2 for the heavy alpha chain. In further embodiments, the monoclonal antibody comprising the sequences according to SEQ ID Nos 3, 4, and 5 for the heavy alpha chain. In further embodiments the monoclonal antibody having the sequence according to SEQ ID No 6 for the light kappa chain. In further embodiments, the monoclonal antibody comprising the sequence according to SEQ ID Nos 7, 8, and 9 for the light kappa chain.

A monoclonal antibody-based biologics in systemic or localized fibrotic diseases to limit the progression of the fibrotic process having the sequences of SEQ ID No 2. and SEQ ID No. 6.

An anti-fibrotic biologic comprising, a full-length chimeric IgG variant, a humanized IgG variant, a scFv variant, or other active biologic including the entire CDRs or their fragments able to bind to the α2Ct target. In further embodiments, the biologic having the sequence according to SEQ ID No 2 for the heavy alpha chain. In further embodiments, the biologic comprising the sequences according to SEQ ID Nos 3, 4, and 5 for the heavy alpha chain. In further embodiments the biologic having the sequence according to SEQ ID No 6 for the light kappa chain. In further embodiments, the biologic comprising the sequence according to SEQ ID Nos 7, 8, and 9 for the light kappa chain.

An antibody fragment comprising a heavy chain comprising CDRs having the sequences: SEQ ID Nos 3, 4, and 5 for the heavy alpha chain and comprising a light chain comprising CDRs having the sequences: SEQ ID Nos 7, 8, and 9 for the light kappa chain.

A single chain antibody comprising CDRs having the sequences: SEQ ID Nos 3, 4, and 5 for the heavy alpha chain.

A single chain antibody comprising a light chain comprising CDRs having the sequences: SEQ ID Nos 7, 8, and 9 for the light kappa chain.

A single chain antibody comprising CDRs having the sequences: SEQ ID Nos 3, 4, and 5 for the heavy alpha chain and comprising a light chain comprising CDRs having the sequences: SEQ ID Nos 7, 8, and 9 for the light kappa chain.

A monoclonal antibody as provided herein, wherein said antibody comprises a further component selected from the group consisting of: a linked polymer, glycosylated, radiolabeled, covalently linked to a moiety, immobilized on a solid support, linked to a toxin, a chemotherapeutic, or an imaging compound; or combinations thereof.

A pharmaceutical composition comprising an antibody having a variable chain of SEQ ID No. 2, and of SEQ ID No. 6.

A method of treating excessive fibrotic tissue formation in a patient comprising administering to said patient an effective amount of a pharmaceutical composition comprising an antibody having a variable chain of SEQ ID No. 2, and of SEQ ID No. 6.

A pharmaceutical composition comprising an antibody having CDR's corresponding to SEQ ID Nos. 3, 4, 5, in the heavy chain and 7, 8, and 9 in the light chain.

A method of treating excessive fibrotic tissue formation in a patient comprising administering to said patient an effective amount of a pharmaceutical composition comprising an antibody having CDR's corresponding to SEQ ID Nos. 3, 4, 5, in the heavy chain and 7, 8, and 9 in the light chain.

A method of limiting growth of fibrotic tissue by blocking collagen fibril formation comprising administering to a patient an effective amount of an anti-fibrotic antibody; wherein the anti-fibrotic antibody comprises a sequence comprising SEQ ID No. 2 and SEQ ID No. 6.

A method of delivering targeted therapeutic compounds to collagen I rich connective tissues comprising administering to a patient an effective amount of an antibody having affinity for collagen I rich tissues, and comprising a therapeutic compound bound to said antibody. A preferred embodiment comprises wherein the therapeutic compound is selected from the group consisting of an antibiotic, a growth factor, therapeutic cells, and a chemotherapeutic agent.

In preferred embodiments an anti-fibrotic antibody can be utilized in the methods described herein wherein the variable region comprises CDR's in a light and heavy chain, comprising SEQ ID Nos. 3, 4, and 5, in the heavy chain and SEQ ID Nos. 7, 8, and 9 in the light chain.

An anti-fibrotic biologic comprising, a full-length chimeric IgG variant, a humanized IgG variant, a scFv variant, or other active biologic including the entire CDRs or their fragments able to bind to the α2Ct target.

In the preferred embodiments, a therapeutic is delivered at the site of excessive fibrosis via systemic deliver, local delivery (injection at wound site), via topical application in the form of an ointment, drops or spray.

### BRIEF DESCRIPTION OF THE DRAWINGS

FIG. 1 schematic of a collagen molecule indicating the target site of the AFA (asterisk). Symbols: Nt, Ct, the N-terminal and the C-terminal telopeptides of collagen I.

FIG. 2 Alignment of the sequences of the V_{H} and the V_{L} of the AFA (upper lines) with homologous regions from other antibodies. Presented examples of antibodies are characterized by the highest identity scores. While the upper lanes represent the sequences of the V regions of the AFA (CDRs highlighted with greyscale) the lower lanes identify the sequences of antibodies from protein data bases. In these lanes the light highlights show regions with identical amino acid sequences while the dark highlights show regions with different amino acid residues.

FIG. 3 mapping of epitopes recognized by the AFA construct.

FIG. 4 depicts kinetics of binding interactions between the ACA and the α2Ct. Association and dissociation data for the full-length and Fab variants are indicated. Based on the kinetics of the association and the dissociation phases, we calculated the K_{D} values for the following binding interactions:

### DEFINITIONS

The terms “antibody” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like. Also encompassed by the term are Fab′, Fv, F(ab′)2, and or other antibody fragments that retain specific binding to antigen, and monoclonal antibodies. An antibody may be monovalent or bivalent.

“Antibody fragments” comprise a portion of an intact antibody, for example, the antigen binding or variable region of the intact antibody. Examples of antibody fragments include Fab, Fab′, F(ab′)2, and Fv fragments; diabodies; linear antibodies (Zapata et al., Protein Eng. 8(10): 1057-1062 (1995)); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2.

“Single-chain Fv” or “sFv” antibody fragments comprise the VH and VL domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-Chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

As used herein, the term “affinity” refers to the equilibrium constant for the reversible binding of two agents and may be expressed as a dissociation constant (Kd). Affinity of an antibody for a specific antigen can be at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. A subject anti-Collagen I (e.g., an anti-Collagen I antibody or antigen-binding fragment) binds specifically to an epitope within a Collagen I polypeptide. Non-specific binding would refer to binding with an affinity of less than about 10-7 M, e.g., binding with an affinity of 10-6 M, 10-5 M, 10-4 M, etc.

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison.

TABLE 1
CDR Definitions

| | | | | | | | |
|---|---|---|---|---|---|---|---|
| (1) | | (2) | Kabat¹ | (3) | Chothia² | (4) | MacCallum³ |
| (5) | V_{H}CDR1 | (6) | 31-35 | (7) | 26-32 | (8) | 30-35 |
| (9) | V_{H}CDR2 | (10) | 50-65 | (11) | 53-55 | (12) | 47-58 |
| (13) | V_{H}CDR3 | (14) | 95-102 | (15) | 96-101 | (16) | 93-101 |
| (17) | V_{L}CDR1 | (18) | 24-34 | (19) | 26-32 | (20) | 30-36 |
| (21) | V_{L}CDR2 | (22) | 50-56 | (23) | 50-52 | (24) | 46-55 |
| (25) | V_{L}CDR3 | (26) | 89-97 | (27) | 91-96 | (28) | 89-96 |

¹Residue numbering follows the nomenclature of Kabat et al., supra.
²Residue numbering follows the nomenclature of Chothia et al., supra.
³Residue numbering follows the nomenclature of MacCallum et al., supra.

The phrase “conservative amino acid substitution” refers to grouping of amino acids on the basis of certain common properties. A functional way to define common properties between individual amino acids is to analyze the normalized frequencies of amino acid changes between corresponding proteins of homologous organisms (Schulz, G. E. and R. H. Schirmer, Principles of Protein Structure, Springer-Verlag). According to such analyses, groups of amino acids may be defined in which amino acids within a group are exchanged preferentially with each other, and therefore resemble each other most in their impact on the overall protein structure (Schulz, G. E. and R. H. Schirmer, Principles of Protein Structure, Springer-Verlag). Examples of amino acid groups defined in this manner include:

(i) a charged group, consisting of Glu and Asp, Lys, Arg and His,

(ii) a positively-charged group, consisting of Lys, Arg and His,

(iii) a negatively-charged group, consisting of Glu and Asp,

(iv) an aromatic group, consisting of Phe, Tyr and Trp,

(v) a nitrogen ring group, consisting of His and Trp,

(vi) a large aliphatic non-polar group, consisting of Val, Leu and Ile,

(vii) a slightly-polar group, consisting of Met and Cys,

(viii) a small-residue group, consisting of Ser, Thr, Asp, Asn, Gly, Ala, Glu, Gin and Pro,

(ix) an aliphatic group consisting of Val, Leu, Ile, Met and Cys, and

(x) a small hydroxyl group consisting of Ser and Thr.

“Homology” or “identity” or “similarity” refers to sequence similarity between two peptides or between two nucleic acid molecules. Homology and identity can each be determined by comparing a position in each sequence which may be aligned for purposes of comparison. When an equivalent position in the compared sequences is occupied by the same base or amino acid, then the molecules are identical at that position; when the equivalent site is occupied by a similar amino acid residue (e.g., similar in steric and/or electronic nature), then the molecules can be referred to as homologous (similar) at that position. Expression of a percentage of homology/similarity or identity refers to a function of the number of identical or similar amino acids at positions shared by the compared sequences. A sequence which is “unrelated” or “non-homologous” shares less than 40% identity, or less than 25% identity, with a reference sequence. In comparing two sequences, the absence of residues (amino acids or nucleic acids) or presence of extra residues also decreases the identity and homology/similarity.

The term “homology” describes a mathematically based comparison of sequence similarities which is used to identify genes or proteins with similar functions or motifs. A reference amino acid (protein) sequence (e.g., a sequence shown herein) may be used as a “query sequence” to perform a search against public databases to, for example, identify other family members, related sequences or homologs. Such searches can be performed using the NB LAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a reference nucleic acid. BLAST amino acid searches can be performed with the XBLAST program, score=50, wordlength=3 to obtain amino acid sequences homologous to a reference amino acid sequence. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used (see ncbi.nlm.nih.gov).

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well-known Smith Waterman algorithm may also be used to determine identity.

The term “substantially identical” means identity between a first amino acid sequence that contains a sufficient or minimum number of amino acid residues that are (i) identical to, or (ii) conservative substitutions of, aligned amino acid residues in a second amino acid sequence such that the first and second amino acid sequences can have a common structural domain and/or common functional activity. For example, amino acid sequences that contain a common structural domain having at least about 90%, 91%, 92%, 93%, 94%, 95%, 96%, 97%, 98% or 99% identity to Collagen I are termed sufficiently or substantially identical to the Collagen I, specifically α2Ct polypeptide. In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc.

A “therapeutically effective amount” or “efficacious amount” refers to the amount of a compound (e.g. a subject antibody) that, when administered to a mammal or other subject for treating a disease, is sufficient to effect such treatment for the disease. The “therapeutically effective amount” will vary depending on the antibody, the disease and its severity and the age, weight, etc., of the subject to be treated.

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosed embodiments. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art of the disclosure. All publications mentioned herein are incorporated herein by reference to disclose and describe the methods and/or materials in connection with which the publications are cited.

As used herein and in the appended claims, the singular forms “a,” “an,” and “the” include plural referents unless the context clearly dictates otherwise. It is noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the presently-claimed subject matter is not entitled to antedate such publication by virtue of prior invention. Further, the dates of publication provided may be different from the actual publication dates which may need to be independently confirmed.

### DETAILED DESCRIPTION OF THE EMBODIMENTS

To date, no effective therapeutics for excessive fibrosis exist. Therefore, there is a need to develop new approaches to inhibit the process of excessive deposition of fibrotic tissue whose main components are collagen fibrils. Employing in vitro and in vivo assays, we demonstrated that the process of excessive deposition of fibrotic tissue can be reduced by inhibiting collagen fibril formation 1-5. The antibody approach to limit fibrosis is attractive because antibody-based therapeutics are generally safe and their in vivo behavior is well understood. Thus, our identifying the CDRs of the AFA and determining specific binding epitopes within the CTTR1 enables engineering of safe and effective human-relevant inhibitors of fibrosis. To that end, we manufactured antibodies, both in full length, Fab, as well as single chain antibodies, having the CDRs of SEQ ID Nos 3-5, and 7-9, wherein the antibodies possess strong binding to α2Ct, both native and synthetic. Accordingly, such antibodies, having strong binding properties, can be utilized for therapeutically targeting and binding to such peptides.

Limitations of current anti-fibrotic strategies: Fibrotic deposits are formed as a result of a cascade-like process that includes inflammation, increased proliferation of specific cells, and biosynthesis of components of the extracellular matrix (ECM). Most of these biological processes are considered potential targets for inhibitors of fibrosis. Thus, these inhibitors aim at (i) reducing inflammation, (ii) blocking cytokines and growth factors that promote fibrosis, (iii) reducing cell proliferation, and (iv) decreasing the biosynthesis of functional collagen molecules at transcription, translation, and posttranslational levels. Because most of the potential targets are involved not only in pathological fibrosis, but also in a number of physiological processes, their inhibition is frequently associated with significant adverse effects. In addition, the majority of current approaches focus on targeting broad upstream cellular processes of the fibrosis cascade, thereby increasing the chance for adverse effects. In contrast, our discovery will allow employing a safe strategy that targets a specific downstream process in this cascade, namely the extracellular formation of collagen fibrils, an approach that limits the chances for adverse effects.

We have demonstrated that binding of the native mouse IgA-type AFA, its chimeric human IgG-type or the scFv variant, all containing the CDRs described here, to the CTTR1 inhibits the formation of collagen fibrils, a main component of fibrotic tissues 1-3; A. Steplewski, et. al, Blocking Collagen Fibril Formation in Injured Knees Reduces Flexion Contracture in a Rabbit Model, J. Orthopaedic Research Society, DOE 10.1002;jor.23369 (Jul. 29, 2016); J. Fertala et al., Target-Specific Delivery of an Antibody That Blocks the Formation of Collagen Deposits in Skin and Lung, Monoclonal Antibodies in Immunodiagnosis and immunotherapty, vol 36 No. 5, 2017. Consequently, employing in vitro and in vivo assays, we demonstrated that CDRs-mediated binding of the AFA variants to the CTTR1 represents a valid antifibrotic approach 1-4. The amino acid sequences of the CDRs of the AFA were obtained by sequencing cDNA derived from mRNA isolated from a hybridoma clone that produces the original mouse IgA-type variant of the AFA. The importance of determining the amino acid sequences of the CDRs of the AFA is that now it is possible to employ the AFA variants with the potential to act as anti-fibrotic therapeutics in humans. Examples of such variants include the following: (i) chimeric mouse/human antibodies consisting of mouse variable regions that include the CDRs identified here and human constant regions of immunoglobulins from the IgG class, (ii) humanized antibodies consisting of the CDRs identified here and human regions of immunoglobulins from the IgG class, and (iii) single-chain antibody that includes the CDRs identified here. We envision that the above variants can be applied at sites of excessive fibrosis via systemic delivery, via local delivery (e.g. injection to the edge of wound), via topical application in a form of ointments (e.g., skin) or drops (e.g., eye), and spray (e.g., lung).

Addressing Current Unmet Need:

Because the current treatments to limit fibrosis are not fully effective, novel approaches have yet to be identified and explored. By defining the sequence of the CDRs that mediate blocking excessive fibrosis, our invention addresses such a need. The impact of our invention will be significant. Since excessive deposition of collagen fibrils is a hallmark of localized and systemic fibrotic changes, inhibiting the collagen fibril formation process via CDRs-mediated blocking of the CTTR1 described here will have a broad positive impact on reducing fibrosis in distinct tissues and organs.

Considering localized fibrotic response, for instance after surgery in the abdomen, the formation of excessive scar tissue around abdominal organs often interferes with the organs' functionality. Moreover, after plastic surgery to the face, the formation of excessive scar tissue frequently compromises the benefits of the surgery. Excessive scar formation also presents a major complication in the eye after glaucoma surgery performed to maintain a lamellar channel from the subconjunctival space to the anterior chamber. Frequently, however, excessive scar formation closes this pressure-reducing channel, thereby forcing the intraocular pressure to rise.

Yet another significant problem with excessive formation of fibrous deposits is the foreign body response to medical devices and materials implanted in the human body. Furthermore, posttraumatic formation of fibrotic scars around joints is a main reason for developing joint stiffness, and fibrotic scarring of segmental defects of peripheral nerves is a main factor that hampers nerve regeneration. Similarly to the above examples of localized fibrosis, fibrotic changes may affect the entire organs including lungs, liver, kidney, and skin. Pathological changes associated with excessive accumulation of collagen fibrils in affected organs alter their function and are a prime reason for organ transplant. Because of such wide tissue distribution of possible fibrotic changes, and the multitude of medical situations in which these changes occur, we expect the impact of the described discovery on developing inhibitors of fibrosis to be high.

A. Sequencing of DNA Fragments Encoding the Variable Regions of the Original Mouse IgA-Type Anti-α2Ct Antibody.

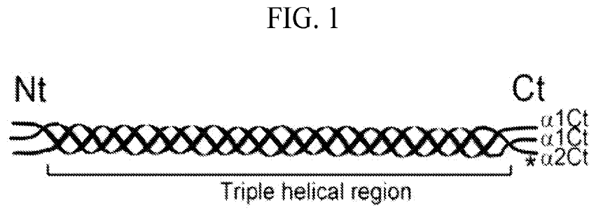

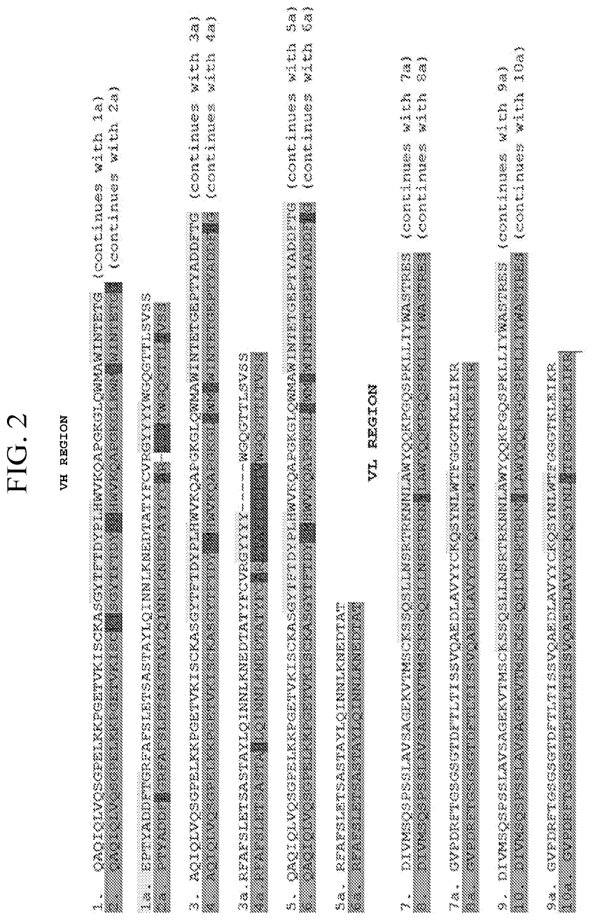

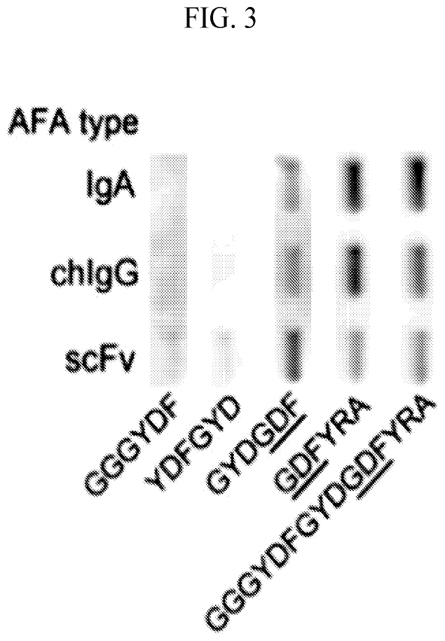

Isolation of RNA from hybridoma cells expressing the original IgA-type anti-α2Ct antibody. Selection of hybridoma cells producing the AFA of the IgA class that recognizes the α2Ct (FIG. 1) and blocks the collagen fibril formation are described elsewhere 1. Total RNA was prepared from hybridoma cells with the use of an RNA-isolation kit according to the manufacturer's protocol (QIAGEN). Sequencing the variable regions of the heavy a chain (VH) and the light κ chain (VL). RNA isolated from hybridoma cells was used as a template to generate PCR products spanning regions encoding the VH or the VL. Sequencing of these PCR products was performed, as described 3. Determining the sequences of CDRs. The CDRs of the variable domains were identified with Rosetta software (http://rosie.graylab.jhu.edu/). Comparing the sequences of the VH and VL sequences to those present in protein databases. Employing the BLAST, we compared the VH and VL sequences to homologous sequences of other antibodies present in the protein databases including the patented protein sequences (FIG. 2).

The Sequence as Listed in FIG. 2 are as Follows:

Sequence 1: VH region of the AFA (SEQ ID No. 2)

Sequence 2: Immunoglobulin heavy chain variable region, partial [Mus musculus]; GenBank: BAA32079.1. (SEQ ID No. 10)

Sequence 3: VH region of the AFA (SEQ ID No. 2)

Sequence 4: Immunoglobulin heavy chain variable region, partial [Mus musculus]; GenBank: AAC37615.1. (SEQ ID No. 11)

Sequence 5: VH region of the AFA (SEQ ID No. 2)

Sequence 6: Ig heavy chain V region (subgroup XI)-mouse (fragment); UniProtKB: locus S24766(SEQ ID No. 12)

Sequence 7: VL region of the AFA (SEQ ID No. 6)

Sequence 8: Anti-meningococcal polysaccharide group C monoclonal antibody 3079.6 immunoglobulin light chain, partial [Mus musculus]; GenBank: AA073036.1 (SEQ ID No. 13)

Sequence 9: VL region of the AFA (SEQ ID No. 6)

Sequence 10: Anti-hemoglobin 2A1 monoclonal antibody immunoglobulin light chain variable region, partial [Mus musculus]; GenBank: ACJ09393.1 (SEQ ID No. 14)

Epitope Binding Characteristics of the AFA.

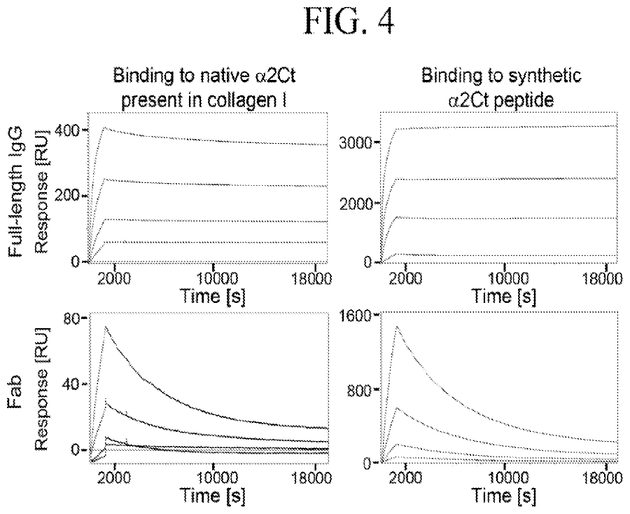

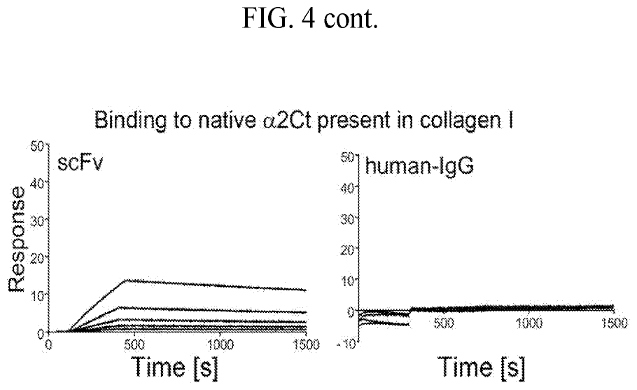

Biosensor assays of binding interactions of the AFA and its Fab fragments with procollagen I and the α2Ct. We analyzed binding between procollagen I and the full-length AFA and between synthetic α2Ct and the full-length AFA. Moreover, we also employed the Fab fragments of the AFA antibody to study their interactions with procollagen I and the α2Ct peptide. FIG. 4 presents results of these assays.

In brief, human procollagen I isolated from human dermal fibroblasts and synthetic α2Ct were immobilized on separate channels of a biosensor. Subsequently, the full-length AFA or its Fab fragments, generated by digestion with papain, were added at various concentrations to a sensor to record the association and the dissociation phases. Data from the AFA binding interactions and the Fab binding interactions were then used to calculate the KD values. In a separate set of experiments, the binding interactions of the scFv variant consisting of the VL and VH domains connected via a peptide linker were also tested using a biosensor. In these assays, the scFv-procollagen I binding interactions were studied.

FIG. 4 depicts the binding kinetics of the following interactions: (i) between the AFA and procollagen I; (ii) between the AFA and the α2Ct; (iii) between the Fab fragment of the AFA and procollagen I; (iv) between the Fab fragment of the AFA and the α2C; (v) between the scFv and procollagen I; and (vi) between non-reactive control human IgG (hIgG) and procollagen I.

TABLE 2
Binding interactions of the AFA and its Fab fragments with native
α2Ct present in procollagen I and with synthetic α2Ct.

| Binding interaction | K_{D} | |
|---|---|---|
| Full-length AFA/procollagen I | 663 | pM |
| Fab AFA antibody/procollagen I | 268 | nM |
| Full-length AFA antibody/synthetic α2Ct | 21 | pM |
| Fab AFA antibody/synthetic α2Ct | 57 | nM |
| scFv/procollagen I | 75 | nM |

These results suggest the following characteristics of the AFA-α2Ct binding: (i) the AFA may bind to the α2Ct peptide by antigen clasping where both Fab domains are engaged in the binding and (ii) native α2Ct present in procollagen I may have more favorable conformation for the AFA binding than its linear synthetic form.

Kinetics of the binding of the AFA to defined α2Ct epitopes. Employing a biosensor, we also analyzed the kinetics of the binding of the AFA with defined epitopes of the human α2Ct. For these assays we employed the AFA, control human IgG, and a set of overlapping peptides spanning the α2Ct (Table 3). In brief, the AFA and control human IgG were covalently immobilized on separate channels of a sensor chip. Subsequently, the binding of the α2Ct fragments to the immobilized antibodies was analyzed. Finally, the dissociation equilibrium constant (K_{D}) values for each α2Ct fragment were calculated (Table 1).

Embodiments of the present disclosure comprising antibodies, Fabs and single chain antibodies, suitable for binding to the α2Ct peptide of Collagen I. These antibodies comprise a heavy chain and a light chain, wherein in the variable regions the CDRs having the sequences: SEQ ID Nos 3, 4, and 5 for the heavy chain, and SEQ ID Nos 7, 8, and 9 for the light chain.

It is suitable, in certain instances to modify antibody, specifically those outside of the CDRs with one or more amino acids. Preferable modifications of these sequences provide homology to the sequence. In certain embodiments, the modifications or differences between a first and second sequence are based upon conservative amino acid substitution, as defined herein, wherein the substitution provides for a similar amino acid exchange. However, homology does not require that the modifications or differences are conservative amino acid substitutions.

TABLE 3
Defining the AFA-α2Ct binding characteristics

| α2Ct fragment | K_{D} |
|---|---|
| GGGYDFGYDGDFYRA (full-length α2Ct) | 21 pM* |
| (SEQ ID No. 1) | |
| GGGYD | 253.7 mM |
| GYDFG | 259 μM |
| DFGYD | 6.4 mM |
| GYDGD | 604 μM |
| DGDFY | 22.2 μM* |
| DFYRA | 449.2 μM |

Results:

Binding of the AFA to the α2Ct fragments—The equilibrium dissociation constant (KD) values for the binding of the AFA to the α2Ct fragments are presented in Table 3. The top sequence in Table 3 is identified as SEQ ID No. 1.

For the first time the presented results describe the KD values for the interaction of the AFA with defined epitopes of the α2Ct. These results indicate that the strongest binding occurs between the AFA and the full-length α2Ct or its DGDFY fragment. Thus, these data suggest that the most critical epitope for the AFA is that containing the GDF sequence. This result supports our earlier observations on the binding of the AFA variants with the biotinylated peptides spanning the α2Ct sequence (FIG. 3).

We observed a relatively strong AFA binding to the native full-length α2Ct present in procollagen I and to the full-length synthetic α2Ct. This binding, however, was significantly weaker to the α2Ct fragments (Table 3). Considering also the Fab binding characteristics (Table 2), the above results suggest the following properties of the AFA-α2Ct binding: (i) The AFA may bind to the α2Ct peptide by antigen clasping where both Fab domains are engaged in the binding; (ii) Native α2Ct present in procollagen I may have more favorable conformation for the AFA binding than its linear synthetic form; (iii) Although the DGDFY epitope has superior AFA-binding characteristics when compared to other fragments of the α2Ct its binding affinity for the AFA is low in comparison to that for the full-length α2Ct (Table 3); (iv) For the high-affinity binding the DGDFY epitope should be, most likely, presented in a context of the α2Ct sequence.

Accordingly, a particular embodiment is directed towards an antibody having a binding characteristic specifically for the DGDFY segment of SEQ ID No. 1, wherein said antibody comprises one binding segment suitable for binding to the DGDFY segment.

Sequences of the PCR products. The PCR products spanning the VH of the α and the VL of the κ chains were sequenced. Below are the amino acid sequences of the variable regions, excluding the signal peptides, with the predicted CDRs highlighted in bold font:

1. VH region:
(SEQ ID No. 2)
QAQIQLVQSGPELKKPGETVKISCKASGYTFTDYPLHWVKQAPGKGLQWM
AWINTETGEPTYADDFTGRFAFSLETSASTAYLQINNLKNEDTATYFCVR
GYYYYWGQGTTLSVSS
SEQ ID No. 3
GYTFTDYPLH;
SEQ ID No. 4
WINTETGEPTYADDFTG;
SEQ ID No. 5
GYYYY
2. VL region:
(SEQ ID NO. 6)
DIVMSQSPSSLAVSAGEKVTMSCKSSQSLLNSRTRKNNLAWYQQKPGQSPK
LLIYWASTRESGVPDRFTGSGSGTDFTLTISSVQAEDLAVYYCKQSYNLWT
FGGGTKLEIKR
SEQ ID No. 7
KSSQSLLNSRTRKNNLA;
SEQ ID No. 8
WASTRES;
SEQ ID NO. 9
KQSYNLWT

FIG. 1 depicts a schematic of a collagen molecule indicating the target site of the AFA (Asterisk). Symbols: Nt, Ct, the N-terminal and the C-terminal telopeptides of collagen I.

FIG. 2 depicts antibodies, and the CDR regions characterized against the highest identify scores. The upper lanes represent the sequences of the V regions of the AFA (CDRs in greyscale, of either SEQ ID No. 2 or 6), the lower lanes identify the sequences of antibodies from protein databases. In these lanes the light greyscale show regions with identical amino acid sequences while the dark greyscale highlights show regions with different amino acid residues. However, even these small changes can modify the binding affinity.

Indeed, as depicted in FIG. 3, we take three antibody types, the IgA, chlgG, and scFv and test for binding. The binding of biotinylated overlapping peptides spanning the α2Ct sequence to the AFA antibody variants immobilized on nitrocellulose membranes was visualized by chemiluminescence. The sequences of employed biotinylated peptides are indicated. As provided above, the underlined GDF sequence represents the critical region recognized by all antibody variants, and thus possession of the GDF sequence enables each different sized antibody to bind, wherein omitting such sequence results in low binding, as shown in the second lane.

Therefore, it is suitable to generate an antibody, for example an IgA, a chlgG, or a scFv antibody, and generate binding when the GDF sequence is conserved. Therefore, a particular embodiment is directed towards an antibody possessing affinity for binding with α2Ct, having a sequence overlapping the GDF sequence in SEQ ID No. 1.

Embodiments—The preferred embodiments comprise an anti-fibrotic antibody (AFA) suitable to limit or block growth of fibrotic tissue by blocking collagen fibril formation. Accordingly, in a preferred embodiment, an antibody, comprising SEQ ID No 2 for the heavy alpha chain for the heavy chain and SEQ ID. No 6 for the light kappa chain is administered to a patient in need thereof.

The antibody administered comprises an amino acid sequence having at least about 90%, at least about 95%, at least about 98%, at least about 99%, or 100%, amino acid sequence identity with SEQ ID Nos 2 and SEQ ID Nos. 6. Or, alternatively with the CDR regions comprising SEQ ID Nos. 3, 4, 5, of the heavy alpha chain and SEQ ID Nos. 7, 8, and 9 for the light kappa chain.

A further embodiment may be for a method of treatment of fibrosis in a patient by administering to said patient an antibody comprising SEQ ID Nos 2 and SEQ ID Nos. 6 for the heavy alpha chain and the light kappa chain. Or, alternatively with the CDR regions comprising SEQ ID Nos. 3, 4, 5, of the heavy alpha chain and SEQ ID Nos. 7, 8, and 9 for the light kappa chain.

A further embodiment is directed to a mechanism for delivering a therapeutic agent to collagen I-rich connective tissues; comprising administering to a patient an antibody comprising SEQ ID Nos 2 and SEQ ID Nos. 6 for the heavy alpha chain and the light kappa chain. Or, alternatively with the CDR regions comprising SEQ ID Nos. 3, 4, 5, of the heavy alpha chain and SEQ ID Nos. 7, 8, and 9 for the light kappa chain.

In certain preferred embodiments, the antibody suitable for treatment in the above methods is a full length, chimeric IgG variant, a humanized IgG variant, an asFv variant, or another active biologic that comprises the CDR's corresponding SEQ ID Nos 2 and SEQ ID Nos. 6 for the heavy alpha chain and the light kappa chain. Or, alternatively with the CDR regions comprising SEQ ID Nos. 3, 4, 5, of the heavy alpha chain and SEQ ID Nos. 7, 8, and 9 for the light kappa chain, which are specifically able to bind to the α2Ct target.

A method of reducing fibrosis formation, comprising administering to a patient an effective amount of a pharmaceutical composition comprising am anti-fibrotic biologic comprising amino acid sequence having at least about 90%, at least about 95%, at least about 98%, at least about 99%, or 100%, amino acid sequence identity with SEQ ID Nos 2 and SEQ ID Nos. 6 for the heavy alpha chain and the light kappa chain. Or, alternatively with the CDR regions comprising SEQ ID Nos. 3, 4, 5, of the heavy alpha chain and SEQ ID Nos. 7, 8, and 9 for the light kappa chain. Preferably the anti-fibrotic biologic is selected from the group consisting of: a full length, chimeric IgG variant, a humanized IgG variant, an asFv variant.

In preferred embodiments, a biologic, preferably an antibody binds to the α2Ct target with an affinity of at least about 10⁻⁵M, at least about 10⁻⁶M, at least about 10⁻⁷M, at least about 10⁻⁸M, at least about 10⁻⁹M, at least about 10⁻¹⁰M, at least about 10⁻¹¹ M, or at least about 10⁻¹² M, or greater than 10⁻¹²M. A subject antibody binds to an epitope present on a α2Ct polypeptide with an affinity of from about 10⁻⁵M to about 10⁻⁶M, 10⁻⁶M to about 10⁻⁷M, 10⁻⁷M to about 10⁻⁸ M, from about 10⁻⁸M to about 10⁻⁹M, from about 10⁻⁹M to about 10⁻¹⁰M, from about 10⁻¹⁰ M to about 10⁻¹¹M, or from about 10⁻¹¹ M to about 10⁻¹²M, or greater than 10⁻¹²M. Examples of the binding affinity are provided in the figures herein.

In certain embodiments, an antibody for binding to the α2Ct target comprises a VH and a VL region, where: 1) the VH region comprises one, two, or three heavy chain variable region CDRs comprising an amino acid sequence that is 85%, 86%, 87%, 88%, 89%, 90%, 91%, 92%, 93%, 94%, 95%, 96%, 97%, 98% or 99% identical to SEQ ID No 2: and 2) the V_{L} region comprises one, two, or three light chain variable region CDRs comprising an amino acid sequence that is 85%, 86%, 87%, 88%, 89%, 90%, 91%, 92%, 93%, 94%, 95%. 96%, 97%, 98% or 99% identical to SEQ ID NO. 6.

Those of skill in the art recognize that antibodies of the present disclosure can be modified to include one or more additional components as described below.

In some embodiments, a subject antibody comprises a free thiol (—SH) group at the carboxyl terminus, where the free thiol group can be used to attach the antibody to a second polypeptide (e.g., another antibody, including a subject antibody), a scaffold, a carrier, etc.

In some embodiments, a subject antibody comprises one or more non-naturally occurring amino acids. In some embodiments, the non-naturally-occurring amino acid comprises a carbonyl group, an acetyl group, an aminooxy group, a hydrazine group, a hydrazide group, a semicarbazide group, an azide group, or an alkyne group. See, e.g., U.S. Pat. No. 7,632,924 for disclosure of exemplary non-naturally occurring amino acids. Inclusion of a non-naturally occurring amino acid can provide for linkage to a polymer, a second polypeptide, a scaffold, etc. For example, a subject antibody linked to a water-soluble polymer can be made by reacting a water-soluble polymer (e.g., PEG) that comprises a carbonyl group to the subject antibody that comprises a non-naturally encoded amino acid that comprises an aminooxy, hydrazine, hydrazide or semicarbazide group. As another example, a subject antibody linked to a water-soluble polymer can be made by reacting a subject antibody that comprises an alkyne-containing amino acid with a water-soluble polymer (e.g., PEG) that comprises an azide moiety; in some embodiments, the azide or alkyne group is linked to the PEG molecule through an amide linkage. A “non-naturally occurring amino acid” refers to an amino acid that is not one of the 20 common amino acids, or pyrolysine or selenocysteine. Other terms that may be used synonymously with the term “non-naturally occurring amino acid” are “non-natural amino acid,” “unnatural amino acid,” “non-naturally-encoded amino acid,” and variously hyphenated and non-hyphenated versions thereof. The term “non-naturally occurring amino acid” also includes, but is not limited to, amino acids that occur by modification (e.g. post-translational modifications) of a naturally encoded amino acid (including but not limited to, the 20 common amino acids or pyrolysine and selenocysteine) but are not themselves naturally incorporated into a growing polypeptide chain by the translation complex. Examples of such non-naturally-occurring amino acids include, but are not limited to, N-acetylglucosaminyl-L-serine, N-acetylglucosaminyl-L-threonine, and O-phosphotyrosine.

In some embodiments, a subject antibody is linked (e.g., covalently linked) to a polymer (e.g., a polymer other than a polypeptide). Suitable polymers include, e.g., biocompatible polymers, and water-soluble biocompatible polymers. Suitable polymers include synthetic polymers and naturally-occurring polymers. Suitable polymers include, e.g., substituted or unsubstituted straight or branched chain polyalkylene, polyalkenylene or polyoxyalkylene polymers or branched or unbranched polysaccharides, e.g. a homo- or hetero-polysaccharide. Suitable polymers include, e.g., ethylene vinyl alcohol copolymer (commonly known by the generic name EVOH or by the trade name EVAL); polybutylmethacrylate; poly(hydroxyvalerate); poly(L-lactic acid); polycaprolactone; poly(lactide-co-glycolide); poly(hydroxybutyrate); poly(hydroxybutyrate-co-valerate); polydioxanone; polyorthoester; polyanhydride; poly(glycolic acid); poly(D,L-lactic acid); poly(glycolic acid-co-trimethylene carbonate); polyphosphoester; polyphosphoester urethane; poly(amino acids); cyanoacrylates; poly(trimethylene carbonate); poly(iminocarbonate); copoly(ether-esters) (e.g., poly(ethylene oxide)-poly(lactic acid) (PEO/PLA) co-polymers); polyalkylene oxalates; polyphosphazenes; biomolecules, such as fibrin, fibrinogen, cellulose, starch, collagen and hyaluronic acid; polyurethanes; silicones; polyesters; polyolefins; polyisobutylene and ethylene-alphaolefin copolymers; acrylic polymers and copolymers; vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile; polyvinyl ketones; polyvinyl aromatics, such as polystyrene; polyvinyl esters, such as polyvinyl acetate; copolymers of vinyl monomers with each other and olefins, such as ethylene-methyl methacrylate copolymers, acrylonitrile-styrene copolymers, acetonitrile butadiene styrene (ABS) resins, and ethylene-vinyl acetate copolymers; polyamides, such as Nylon 66 and polycaprolactam; alkyd resins; polycarbonates; polyoxymethylenes; polyimides; polyethers; epoxy resins; polyurethanes; rayon; rayon-triacetate; cellulose; cellulose acetate; cellulose butyrate; cellulose acetate butyrate; cellophane; cellulose nitrate; cellulose propionate; cellulose ethers; amorphous Teflon; poly(ethylene glycol); and carboxymethyl cellulose.

Suitable synthetic polymers include unsubstituted and substituted straight or branched chain poly(ethyleneglycol), poly(propyleneglycol) poly(vinylalcohol), and derivatives thereof, e.g., substituted poly(ethyleneglycol) such as methoxypoly(ethyleneglycol), and derivatives thereof. Suitable naturally-occurring polymers include, e.g., albumin, amylose, dextran, glycogen, and derivatives thereof.

Suitable polymers can have an average molecular weight in a range of from 500 Da to 50,000 Da, e.g., from 5,000 Da to 40,000 Da, or from 25,000 to 40,000 Da. For example, in some embodiments, in which a subject antibody comprises a poly(ethylene glycol) (PEG) or methoxypoly(ethyleneglycol) polymer, the PEG or methoxypoly(ethyleneglycol) polymer can have a molecular weight in a range of from about 0.5 kiloDaltons (kDa) to 1 kDa, from about 1 kDa to 5 kDa, from 5 kDa to 10 kDa, from 10 kDa to 25 kDa, from 25 kDa to 40 kDa, or from 40 kDa to 60 kDa.

As noted above, in some embodiments, a subject antibody is covalently linked to a PEG polymer. In some embodiments, a subject scFv multimer is covalently linked to a PEG polymer. See, e.g., Albrecht et al. (2006) J. Immunol. Methods 310:100. Methods and reagents suitable for PEGylation of a protein are well known in the art and may be found in, e.g., U.S. Pat. No. 5,849,860. PEG suitable for conjugation to a protein is generally soluble in water at room temperature, and has the general formula R(O—CH₂—CH₂)ₙO—R, where R is hydrogen or a protective group such as an alkyl or an alkanol group, and where n is an integer from 1 to 1000. Where R is a protective group, it generally has from 1 to 8 carbons.

The PEG conjugated to the subject antibody can be linear. The PEG conjugated to the subject protein may also be branched. Branched PEG derivatives include, for example, those described in U.S. Pat. No. 5,643,575, “star-PEG's” and multi-armed PEG's such as those described in Shearwater Polymers, Inc. catalog “Polyethylene Glycol Derivatives 1997-1998.” Star PEGs are described in the art including, e.g., in U.S. Pat. No. 6,046,305.

A subject antibody can be glycosylated, e.g., can comprise a covalently linked carbohydrate or polysaccharide moiety. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to an antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites). Similarly, removal of glycosylation sites can be accomplished by amino acid alteration within the native glycosylation sites of an antibody.

A subject antibody will in some embodiments comprise a “radiopaque” label, e.g. a label that can be easily visualized using for example x-rays. Radiopaque materials are well known to those of skill in the art. The most common radiopaque materials include iodide, bromide or barium salts. Other radiopaque materials are also known and include, but are not limited to organic bismuth derivatives (see, e.g., U.S. Pat. No. 5,939,045), radiopaque multiurethanes (see U.S. Pat. No. 5,346,981), organobismuth composites (see, e.g., U.S. Pat. No. 5,256,334), radiopaque barium multimer complexes (see, e.g., U.S. Pat. No. 4,866,132), and the like.

A subject antibody can be covalently linked to a second moiety (e.g., a lipid, a polypeptide other than a subject antibody, a synthetic polymer, a carbohydrate, and the like) using for example, glutaraldehyde, a homobifunctional cross-linker, or a heterobifunctional cross-linker. Glutaraldehyde cross-links polypeptides via their amino moieties. Homobifunctional cross-linkers (e.g., a homobifunctional imidoester, a homobifunctional N-hydroxysuccinimidyl (NHS) ester, or a homobifunctional sulfhydryl reactive cross-linker) contain two or more identical reactive moieties and can be used in a one-step reaction procedure in which the cross-linker is added to a solution containing a mixture of the polypeptides to be linked. Homobifunctional NHS ester and imido esters cross-link amine containing polypeptides. In a mild alkaline pH, imido esters react only with primary amines to form imidoamides, and overall charge of the cross-linked polypeptides is not affected. Homobifunctional sulfhydryl reactive cross-linkers includes bismaleimidhexane (BMH), 1,5-difluoro-2,4-dinitrobenzene (DFDNB), and 1,4-di-(3′,2′-pyridyldithio) propinoamido butane (DPDPB).

Heterobifunctional cross-linkers have two or more different reactive moieties (e.g., amine reactive moiety and a sulfhydryl-reactive moiety) and are cross-linked with one of the polypeptides via the amine or sulfhydryl reactive moiety, then reacted with the other polypeptide via the non-reacted moiety. Multiple heterobifunctional haloacetyl cross-linkers are available, as are pyridyl disulfide cross-linkers. Carbodiimides are a classic example of heterobifunctional cross-linking reagents for coupling carboxyls to amines, which results in an amide bond.

A subject antibody can be immobilized on a solid support. Suitable supports are well known in the art and comprise, inter alia, commercially available column materials, polystyrene beads, latex beads, magnetic beads, colloid metal particles, glass and/or silicon chips and surfaces, nitrocellulose strips, nylon membranes, sheets, duracytes, wells of reaction trays (e.g., multi-well plates), plastic tubes, etc. A solid support can comprise any of a variety of substances, including, e.g., glass, polystyrene, polyvinyl chloride, polypropylene, polyethylene, polycarbonate, dextran, nylon, amylose, natural and modified celluloses, polyacrylamides, agaroses, and magnetite. Suitable methods for immobilizing a subject antibody onto a solid support are well known and include, but are not limited to ionic, hydrophobic, covalent interactions and the like. Solid supports can be soluble or insoluble, e.g., in aqueous solution. In some embodiments, a suitable solid support is generally insoluble in an aqueous solution.

A subject antibody will in some embodiments comprise a detectable label. Suitable detectable labels include any composition detectable by spectroscopic, photochemical, biochemical, immunochemical, electrical, optical or chemical means. Suitable labels include, but are not limited to, magnetic beads (e.g. Dynabeads™), fluorescent dyes (e.g., fluorescein isothiocyanate, texas red, rhodamine, a green fluorescent protein, a red fluorescent protein, a yellow fluorescent protein, and the like), radiolabels (e.g., ³H, ¹²⁵I, ³⁵S, ¹⁴C or ³²P), enzymes (e.g., horseradish peroxidase, alkaline phosphatase, luciferase, and others commonly used in an enzyme-linked immunosorbent assay (ELISA)), and colorimetric labels such as colloidal gold or colored glass or plastic (e.g. polystyrene, polypropylene, latex, etc.) beads.

In some embodiments, a subject antibody comprises a contrast agent or a radioisotope, wherein the contrast agent or radioisotope is one that is suitable for use in imaging, e.g., imaging procedures carried out on humans. Non-limiting examples of labels include radioisotope such as ¹²³I (iodine), ¹⁸F (fluorine), ⁹⁹Tc (technetium), ¹¹¹In (indium), and ⁶⁷Ga (gallium), and contrast agent such as gadolinium (Gd), dysprosium, and iron. Radioactive Gd isotopes (¹⁵³Gd) also are available and suitable for imaging procedures in non-human mammals. A subject antibody can be labeled using standard techniques. For example, a subject antibody can be iodinated using chloramine T or 1,3,4,6-tetrachloro-3α,6α-dephenylglycouril. For fluorination, fluorine is added to a subject antibody by a fluoride ion displacement reaction. See. Muller-Gartner, H., TIB Tech., 16:122-130 (1998) and Saji, H., Crit. Rev. Ther. Drug Carrier Syst., 16(2):209-244 (1999) for a review of synthesis of proteins with such radioisotopes. A subject antibody can also be labeled with a contrast agent through standard techniques. For example, a subject antibody can be labeled with Gd by conjugating low molecular Gd chelates such as Gd diethylene triamine pentaacetic acid (GdDTPA) or Gd tetraazacyclododecane tetraacetic (GdDOTA) to the antibody. See, Caravan et al., Chem. Rev. 99:2293-2352 (1999) and Lauffer et al., J. Magn. Reson. Imaging, 3:11-16 (1985). A subject antibody can be labeled with Gd by, for example, conjugating polylysine-Gd chelates to the antibody. See, for example, Curtet et al., Invest. Radiol., 33(10):752-761 (1998). Alternatively, a subject antibody can be labeled with Gd by incubating paramagnetic polymerized liposomes that include Gd chelator lipid with avidin and biotinylated antibody. See, for example, Sipkins et al., Nature Med., 4:623-626 (1998).

Suitable fluorescent proteins that can be linked to a subject antibody include, but are not limited to, a green fluorescent protein from Aequoria victoria or a mutant or derivative thereof e.g., as described in U.S. Pat. Nos. 6,066,476; 6,020,192; 5,985,577; 5,976,796; 5,968,750; 5,968,738; 5,958,713; 5,919,445; 5,874,304; e.g., Enhanced GFP. Many such GFP are available commercially, e.g., from Clontech, Inc. Additional fluorescent proteins include a red fluorescent protein; a yellow fluorescent protein; and any of a variety of fluorescent and colored proteins from Anthozoan species, as described in, e.g., Matz et al. (1999) Nature Biotechnol. 17:969-973; and the like.

A subject antibody will in some embodiments be linked (e.g., covalently or non-covalently linked) to a fusion partner, e.g., a ligand; an epitope tag; a peptide; a protein other than an antibody; and the like. Suitable fusion partners include peptides and polypeptides that confer enhanced stability in vivo (e.g., enhanced serum half-life); provide ease of purification such as polyhistidine sequences, e.g., 6His (HHHHHH, SEQ ID NO:4), and the like; provide for secretion of the fusion protein from a cell; provide an epitope tag, e.g., GST, hemagglutinin and the like; provide a detectable signal, e.g., an enzyme that generates a detectable product (e.g., β-galactosidase, luciferase, beta-glucuronidase), or a protein that is itself detectable, e.g., a green fluorescent protein, a red fluorescent protein, a yellow fluorescent protein, etc.; provides for multimerization, e.g., a multimerization domain such as an Fc portion of an immunoglobulin; and the like.

The fusion may also include an affinity domain, including peptide sequences that can interact with a binding partner, e.g., such as one immobilized on a solid support, useful for identification or purification. Consecutive single amino acids, such as histidine, when fused to a protein, can be used for one-step purification of the fusion protein by high affinity binding to a resin column, such as nickel sepharose. Exemplary affinity domains include chitin binding domain, S-peptide, T7 peptide, SH2 domain, C-end RNA tag, metal binding domains, e.g., zinc binding domains or calcium binding domains such as those from calcium-binding proteins, e.g., calmodulin, troponin C, calcineurin B, myosin light chain, recoverin, S-modulin, visinin, visinin-like protein, neurocalcin, hippocalcin, frequenin, caltractin, calpain large-subunit, S100 proteins, parvalbumin, calbindin D9K, calbindin D28K, and calretinin, inteins, biotin, streptavidin, MyoD, leucine zipper sequences, and maltose binding protein.

A subject antibody will in some embodiments be fused to a polypeptide that binds to an endogenous blood brain barrier (BBB) receptor. Linking a subject antibody to a polypeptide that binds to an endogenous BBB receptor facilitates crossing the BBB, e.g., in a subject treatment method (see below) involving administration of a subject antibody to an individual in need thereof. Suitable polypeptides that bind to an endogenous BBB include antibodies, e.g., monoclonal antibodies, or antigen-binding fragments thereof, that specifically bind to an endogenous BBB receptor. Suitable endogenous BBB receptors include, but are not limited to, an insulin receptor, a transferrin receptor, a leptin receptor, a lipoprotein receptor, and an insulin-like growth factor receptor. See, e.g., U.S. Patent Publication No. 2009/0156498.

In some embodiments, a subject antibody comprises a polyamine modification. Polyamine modification of a subject antibody enhances permeability of the modified antibody at the BBB. A subject antibody can be modified with polyamines that are either naturally occurring or synthetic. See, for example, U.S. Pat. No. 5,670,477. Useful naturally occurring polyamines include putrescine, spermidine, spermine, 1,3-deaminopropane, norspermidine, syn-homospermidine, thermine, thermospermine, caldopentamine, homocaldopentamine, and canavalmine. Putrescine, spermidine and spermine are particularly useful. Synthetic polyamines are composed of the empirical formula C_{X}H_{Y}N_{Z}, can be cyclic or acyclic, branched or unbranched, hydrocarbon chains of 3-12 carbon atoms that further include 1-6 NR or N(R)₂ moieties, wherein R is H, (C₁-C₄) alkyl, phenyl, or benzyl. Polyamines can be linked to an antibody using any standard crosslinking method.

In some embodiments, a subject antibody is modified to include a carbohydrate moiety, where the carbohydrate moiety can be covalently linked to the antibody. In some embodiments, a subject antibody is modified to include a lipid moiety, where the lipid moiety can be covalently linked to the antibody. Suitable lipid moieties include, e.g., an N-fatty acyl group such as N-lauroyl, N-oleoyl, etc.; a fatty amine such as dodecyl amine, oleoyl amine, etc.; a C₃-C₁₆ long-chain aliphatic lipid; and the like. See, e.g., U.S. Pat. No. 6,638,513. In some embodiments, a subject antibody is incorporated into a liposome.

In some embodiments, a subject anti-Collagen I antibody is conjugated or linked to a therapeutic and/or imaging/detectable moiety. Methods for conjugating or linking antibodies are well known in the art. Associations between antibodies and labels include any means known in the art including, but not limited to, covalent and non-covalent interactions.

In one non-limiting embodiment, a subject anti-Collagen I antibody can be associated with a toxin, a radionuclide, an iron-related compound, a dye, an imaging reagent, a fluorescent label or a chemotherapeutic agent that would be toxic when delivered to a cancer cell. Alternatively, a subject anti-Collagen I antibody can be associated with detectable label, such as a radionuclide, iron-related compound, a dye, an imaging agent or a fluorescent agent for immunodetection of target antigens.

Non-limiting examples of radiolabels include:

³²P, ³³P, ⁴³K, ⁵²Fe, ⁵⁷Co, ⁶⁴Cu, ⁶⁷Ga, ⁶⁷Cu, ⁶⁸Ga, ⁷¹Ge, ⁷⁷Br, ⁷⁶Br, ⁷⁷Br, ⁷⁷As, ⁷⁷Br, ⁸¹Rb/^{81m}Kr, ^{87M} Sr, ⁹⁰Y, ⁹⁷Ru, ⁹⁹Tc, ¹⁰⁰Pd, ¹⁰¹Rb, ¹⁰³Pb, ¹⁰⁵Rb, ¹⁰⁹Pd, ¹¹¹Ag, ¹¹¹In, ¹¹³In, ¹¹⁹Sb, ¹²¹Sn, ¹²³I, ₁₂₅I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Ho, ¹⁶⁹Eu, ¹⁷⁷Lu, ¹⁸⁶Re, ¹⁸⁸Re, ¹⁹¹Os, ¹⁹³Pt, ₁₉₄Ir, ₁₉₇Hg, ¹⁹⁹Au, ²⁰³Pb, ²¹¹At, ²¹²Pb, ²¹²Bi, and ²¹³Bi.

Non-limiting examples of toxins include, for example, diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, tricothecenes, Clostridium perfringens phospholipase C (PLC), bovine pancreatic ribonuclease (BPR), antiviral protein (PAP), abrin, cobra venom factor (CVF), gelonin (GEL), saporin (SAP), and viscumin.

Non-limiting examples of iron-related compounds include, for example, magnetic iron-oxide particles, ferric or ferrous particles, Fe²⁰³ and Fe³⁰⁴. Iron-related compounds and Methods of labeling polypeptides, proteins and peptides can be found, for example, in U.S. Pat. Nos. 4,101,435 and 4,452,773.

In certain embodiments, a subject antibody can be covalently or non-covalently coupled to a cytotoxin or other cell proliferation inhibiting compound, in order to localize delivery of that agent to a tumor cell. For instance, the agent can be selected from: alkylating agents, enzyme inhibitors, proliferation inhibitors, lytic agents, DNA- or RNA-synthesis inhibitors, membrane permeability modifiers, DNA metabolites, dichloroethylsulfide derivatives, protein production inhibitors, ribosome inhibitors, inducers of apoptosis, and neurotoxins.

In certain embodiments, the subject antibodies can be coupled with an agent useful in imaging tumors. Such agents include: metals; metal chelators; lanthanides; lanthanide chelators; radiometals; radiometal chelators; positron-emitting nuclei; microbubbles (for ultrasound); liposomes; molecules microencapsulated in liposomes or nanospheres; monocrystalline iron oxide nanocompounds; magnetic resonance imaging contrast agents; light absorbing, reflecting and/or scattering agents; colloidal particles; fluorophores, such as near-infrared fluorophores. In many embodiments, such secondary functionality/moiety will be relatively large, e.g., at least 25 atomic mass units (amu) in size, and in many instances can be at least 50,100 or 250 amu in size.

In certain embodiments, the secondary functionality is a chelate moiety for chelating a metal, e.g., a chelator for a radiometal or paramagnetic ion. In additional embodiments, it is a chelator for a radionuclide useful for radiotherapy or imaging procedures. Conditions under which a chelator will coordinate a metal are described, for example, by Gasnow et al. U.S. Pat. Nos. 4,831,175, 4,454,106 and 4,472,509, each of which is incorporated herein by reference. As used herein, “radionuclide” and “radiolabel” are interchangeable.

Radionuclides suitable for inclusion in a subject anti-Collagen I antibody include gamma-emitters, positron-emitters, Auger electron-emitters, X-ray emitters and fluorescence-emitters. In some embodiments, beta- or alpha-emitters are used. Examples of radionuclides useful as toxins in radiation therapy include:

³²P, ³³P, ⁴³K, ⁵²Fe, ⁵⁷Co, ⁶⁴Cu, ⁶⁷Ga, ⁶⁷Cu, ⁶⁸Ga, ⁷¹Ge, ⁷⁵Br, ⁷⁶Br, ⁷⁷Br, ⁷⁷As, ⁷⁷Br, ⁸¹Rb/⁸¹MKr, ⁸⁷MSr, ⁹⁰Y, ⁹⁷Ru, ⁹⁹Tc, ¹⁰⁰Pd, ¹⁰¹Rh, ¹⁰³Pb, ¹⁰⁵Rh, ¹⁰⁹Pd, ¹¹¹Ag, ¹¹¹In, ¹¹³In, ¹¹⁹Sb, ¹²¹Sn, ¹²³I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁹Au, ²⁰³Pb, ²¹¹At, ²¹²Pb, ²¹²Bi and ²¹³Bi. Exemplary therapeutic radionuclides include ¹⁸⁸Re, ¹⁸⁶Re, ²⁰³Pb, ²¹²Pb, ²¹²Bi, ¹⁰⁹Pd, ⁶⁴Cu, ⁶⁷Cu, ⁹⁰Y, ¹²⁵I, ¹³¹I, ⁷⁷Br, ²¹¹At, ⁹⁷Ru, ¹⁰⁵Rh, ¹⁹⁸Au and ¹⁹⁹Ag, ¹⁶⁶Ho or ¹⁷⁷Lu.⁹⁹

Tc is a particularly attractive radioisotope for diagnostic applications, as it is readily available to all nuclear medicine departments, is inexpensive, gives minimal patient radiation doses, and has ideal nuclear imaging properties. It has a half-life of six hours which means that rapid targeting of a technetium-labeled antibody is desirable. Accordingly, in certain embodiments, a subject antibody is modified to include a chelating agent for technium.

In still other embodiments, the secondary functionality can be a radiosensitizing agent, e.g., a moiety that increases the sensitivity of cells to radiation. Examples of radiosensitizing agents include nitroimidazoles, metronidazole and misonidazole (see: DeVita, V. T. in Harrison's Principles of Internal Medicine, p. 68, McGraw-Hill Book Co., NY, 1983, which is incorporated herein by reference). The modified antibodies that comprise a radiosensitizing agent as the active moiety are administered and localize at the target cell. Upon exposure of the individual to radiation, the radiosensitizing agent is “excited” and causes the death of the cell.

There is a wide range of moieties which can serve as chelators and which can be derivatized to a subject antibody. For instance, the chelator can be a derivative of 1,4,7,10-tetraazacyclododecanetetraacetic acid (DOTA), ethylenediaminetetraacetic acid (EDTA), diethylenetriaminepentaacetic acid (DTPA) and 1-p-Isothiocyanato-benzyl-methyl-diethylenetriaminepentaacetic acid (ITC-MX). These chelators typically have groups on the side chain by which the chelator can be used for attachment to subject antagonists. Such groups include, e.g., benzylisothiocyanate, by which the DOTA, DTPA or EDTA can be coupled to, e.g., an amine group.

In one embodiment, the chelate moiety is an “NxSy” chelate moiety. As defined herein, the “NxSy chelates” include bifunctional chelators that are capable of coordinately binding a metal or radiometal and, may have N2S2 or N3S cores. Exemplary NxSy chelates are described, e.g., in Fritzberg et al. (1998) PNAS 85: 4024-29; and Weber et al. (1990) Chem. 1: 431-37; and in the references cited therein.

In some embodiments, a subject anti-Collagen I antibody is modified to include a chemotherapeutic agent, e.g., a chemotherapeutic agent is covalently or non-covalently linked to a subject anti-Collagen I antibody.

Chemotherapeutic agents (“chemotherapeutics”) suitable for use in modifying a subject antibody include small chemical entities produced by chemical synthesis. Chemotherapeutics include cytotoxic and cytostatic drugs. Chemotherapeutics may include those which have other effects on cells such as reversal of the transformed state to a differentiated state or those which inhibit cell replication. Examples of known cytotoxic agents suitable for use are listed, for example, in Goodman et al., “The Pharmacological Basis of Therapeutics,” Sixth Edition, A. B. Gilman et al., eds./Macmillan Publishing Co. New York, 1980. These include taxanes, such as paclitaxel and docetaxel; nitrogen such as mechlorethamine, melphalan, uracil mustard and chlorambucil; ethylenimine derivatives, such as thiotepa; alkyl sulfonates, such as busulfan; nitrosoureas, such as lomustine, semustine and streptozocin; triazenes, such as dacarbazine; folic acid analogs, such as methotrexate; pyrimidine analogs, such as fluorouracil, cytarabine and azaribine; purine analogs, such as mercaptopurine and thioguanine; vinca alkaloids, such as vinblastine and vincristine; antibiotics, such as dactinomycin, daunorubicin, doxorubicin, and mitomycin; enzymes, such as platinum coordination complexes, such as cisplatin; substituted urea, such as hydroxyurea; methyl hydrazine derivatives, such as procarbazine; adrenocortical suppressants, such as mitotane; hormones and antagonists, such as adrenocortisteroids (prednisone), progestins (hydroxyprogesterone caproate, acetate and megestrol acetate), estrogens (diethylstilbestrol and ethinyl estradiol), and androgens (testosterone propionate and fluoxymesterone).

In some embodiments, a subject anti-Collagen I antibody is modified to include a chemotherapeutic agent that interferes with protein synthesis. Drugs that interfere with protein synthesis include, e.g., puromycin, cycloheximide, and ribonuclease.

Most of the chemotherapeutic agents currently in use in treating cancer possess functional groups that are amenable to chemical cross-linking directly with an amine or carboxyl group of a subject antibody. For example, free amino groups are available on methotrexate, doxorubicin, daunorubicin, cytosinarabinoside, bleomycin, fludarabine, and cladribine while free carboxylic acid groups are available on methotrexate, melphalan and chlorambucil.

These functional groups, that is free amino and carboxyl groups, are targets for a variety of homobifunctional and heterobifunctional chemical cross-linking agents which can crosslink these drugs directly to, e.g., a free amino group of a subject antibody.

Chemotherapeutic agents contemplated for modification of a subject antibody also include other chemotherapeutic drugs that are commercially available. Merely to illustrate, the chemotherapeutic can be an inhibitor of chromatin function, a DNA damaging agent, an antimetabolite (such as folate antagonists, pyrimidine analogs, purine analogs, and sugar-modified analogs), a DNA synthesis inhibitor, a DNA interactive agent (such as an intercalating agent), or a DNA repair inhibitor.

Methods of Producing Antibodies

A subject antibody can be produced by any known method, e.g., conventional synthetic methods for protein synthesis; recombinant DNA methods; etc.

For those embodiments in which a subject antibody is a single chain polypeptide, it can synthesized using standard chemical peptide synthesis techniques. Where a polypeptide is chemically synthesized, the synthesis may proceed via liquid-phase or solid-phase. Solid phase polypeptide synthesis (SPPS), in which the C-terminal amino acid of the sequence is attached to an insoluble support followed by sequential addition of the remaining amino acids in the sequence, is an example of a suitable method for the chemical synthesis of a subject antibody. Various forms of SPPS, such as Fmoc and Boc, are available for synthesizing a subject antibody. Techniques for solid phase synthesis are described by Barany and Merrifield, Solid-Phase Peptide Synthesis; pp. 3-284 in The Peptides: Analysis, Synthesis, Biology. Vol. 2: Special Methods in Peptide Synthesis, Part A., Merrifield, et al. J. Am. Chem. Soc., 85: 2149-2156 (1963); Stewart et al., Solid Phase Peptide Synthesis, 2nd ed. Pierce Chem. Co., Rockford, Ill. (1984); and Ganesan A. 2006 Mini Rev. Med. Chem. 6:3-10 and Camarero J A et al. 2005 Protein Pept Lett. 12:723-8. Briefly, small insoluble, porous beads are treated with functional units on which peptide chains are built. After repeated cycling of coupling/deprotection, the free N-terminal amine of a solid-phase-attached peptide is coupled to a single N-protected amino acid unit. This unit is then deprotected, revealing a new N-terminal amine to which a further amino acid may be attached. The peptide remains immobilized on the solid-phase and undergoes a filtration process before being cleaved off

Standard recombinant methods can be used for production of a subject antibody. For example, nucleic acids encoding light and heavy chain variable regions, optionally linked to constant regions, are inserted into expression vectors. The light and heavy chains can be cloned in the same or different expression vectors. The DNA segments encoding immunoglobulin chains are operably linked to control sequences in the expression vector(s) that ensure the expression of immunoglobulin polypeptides. Expression control sequences include, but are not limited to, promoters (e.g., naturally-associated or heterologous promoters), signal sequences, enhancer elements, and transcription termination sequences. The expression control sequences can be eukaryotic promoter systems in vectors capable of transforming or transfecting eukaryotic host cells (e.g., COS or CHO cells). Once the vector has been incorporated into the appropriate host, the host is maintained under conditions suitable for high level expression of the nucleotide sequences, and the collection and purification of the antibodies.

Because of the degeneracy of the genetic code, a variety of nucleic acid sequences can encode each immunoglobulin amino acid sequence. The desired nucleic acid sequences can be produced by de novo solid-phase DNA synthesis, by polymerase chain reaction (PCR), or by mutagenesis of an earlier prepared variant of the desired polynucleotide. Oligonucleotide-mediated mutagenesis is an example of a suitable method for preparing substitution, deletion and insertion variants of target polypeptide DNA. See Adelman et al., DNA 2:183 (1983). Briefly, the target polypeptide DNA is altered by hybridizing an oligonucleotide encoding the desired mutation to a single-stranded DNA template. After hybridization, a DNA polymerase is used to synthesize an entire second complementary strand of the template that incorporates the oligonucleotide primer, and encodes the selected alteration in the target polypeptide DNA.

Suitable expression vectors are typically replicable in the host organisms either as episomes or as an integral part of the host chromosomal DNA. Commonly, expression vectors contain selection markers (e.g., ampicillin-resistance, hygromycin-resistance, tetracycline resistance, kanamycin resistance or neomycin resistance) to permit detection of those cells transformed with the desired DNA sequences.

Escherichia coli is an example of a prokaryotic host cell that can be used for cloning a subject antibody-encoding polynucleotide. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters will typically control expression, optionally with an operator sequence, and have ribosome binding site sequences and the like, for initiating and completing transcription and translation.

Other microbes, such as yeast, are also useful for expression. Saccharomyces (e.g., S. cerevisiae) and Pichia are examples of suitable yeast host cells, with suitable vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

In addition to microorganisms, mammalian cells (e.g., mammalian cells grown in in vitro cell culture) can also be used to express and produce a subject antibody. See Winnacker, From Genes to Clones, VCH Publishers, N.Y., N.Y. (1987). Suitable mammalian host cells include CHO cell lines, various COS cell lines, HeLa cells, myeloma cell lines, and transformed B-cells or hybridomas. Expression vectors for these cells can include expression control sequences, such as an origin of replication, a promoter, and an enhancer (Queen et al., Immunol. Rev. 89:49 (1986)), and necessary processing information sites, such as ribosome binding sites, RNA splice sites, polyadenylation sites, and transcriptional terminator sequences. Examples of suitable expression control sequences are promoters derived from immunoglobulin genes, SV40, adenovirus, bovine papilloma virus, cytomegalovirus and the like. See Co et al., J. Immunol. 148:1149 (1992).

Once synthesized (either chemically or recombinantly), the whole antibodies, their dimers, individual light and heavy chains, or other forms of a subject antibody (e.g., scFv, etc.) can be purified according to standard procedures of the art, including ammonium sulfate precipitation, affinity columns, column chromatography, high performance liquid chromatography (HPLC) purification, gel electrophoresis, and the like (see generally Scopes, Protein Purification (Springer-Verlag, N.Y., (1982)). A subject antibody can be substantially pure, e.g., at least about 80% to 85% pure, at least about 85% to 90% pure, at least about 90% to 95% pure, or 98% to 99%, or more, pure, e.g., free from contaminants such as cell debris, macromolecules other than a subject antibody, etc.

Compositions

The present disclosure provides a composition comprising a subject antibody. A subject antibody composition can comprise, in addition to a subject antibody, one or more of: a salt, e.g., NaCl, MgCl, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (YMS), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a protease inhibitor; glycerol; and the like.

The present disclosure provides compositions, including pharmaceutical compositions, comprising a subject antibody. In general, a composition comprises an effective amount of a subject antibody. An “effective amount” means a dosage sufficient to produce a desired result, e.g., reduction in cancer cell number, tumor size, etc., amelioration of a symptom of cancer or a fibrotic disease. Generally, the desired result is at least a reduction in a symptom of cancer or a fibrotic disorder, as compared to a control. A subject antibody can be delivered in such a manner as to avoid the blood-brain barrier, as described in more detail below. A subject antibody can be formulated and/or modified to enable the antibody to cross the blood-brain barrier.

A particular embodiment is directed towards a pharmaceutical composition comprising an antibody having a variable chain of SEQ ID No. 2, and of SEQ ID No. 6. Said pharmaceutical composition may further comprise a buffer and a solubilizing agent, suitable for delivery to a mammal, wherein the pharmaceutical composition is administered in an effective amount.

A particular embodiment is directed towards a method of treating excessive fibrotic tissue formation in a patient comprising administering to said patient an effective amount of a pharmaceutical composition comprising a variable chain of SEQ ID No. 2, and of SEQ ID No. 6. In certain embodiments, the variable chain comprises CDR's corresponding to SEQ ID Nos. 3, 4, 5, in the heavy chain and 7, 8, and 9 in the light chain.

Formulations

In the subject methods, a subject antibody can be administered to the host using any convenient means capable of resulting in the desired therapeutic effect or diagnostic effect. Thus, the agent can be incorporated into a variety of formulations for therapeutic administration. More particularly, a subject antibody can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, a subject antibody can be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, a subject antibody can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

A subject antibody can be formulated into preparations for injection by dissolving, suspending or emulsifying it in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions comprising a subject antibody are prepared by mixing the antibody having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration; see also Chen (1992) Drug Dev Ind Pharm 18, 1311-54.

Exemplary antibody concentrations in a subject pharmaceutical composition may range from about 1 mg/mL to about 200 mg/ml or from about 50 mg/mL to about 200 mg/mL, or from about 150 mg/mL to about 200 mg/mL.

An aqueous formulation of the antibody may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included in the antibody formulation to modulate the tonicity of the formulation. Exemplary tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 nM.

A surfactant may also be added to the antibody formulation to reduce aggregation of the formulated antibody and/or minimize the formation of particulates in the formulation and/or reduce adsorption. Exemplary surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Exemplary concentrations of surfactant may range from about 0.001% to about 1% w/v.

A lyoprotectant may also be added in order to protect the labile active ingredient (e.g. a protein) against destabilizing conditions during the lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some embodiments, a subject formulation includes a subject anti-Collagen I antibody, and one or more of the above-identified agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

For example, a subject formulation can be a liquid or lyophilized formulation suitable for parenteral administration, and can comprise about 1 mg/mL to about 200 mg/mL of a subject antibody; about 0.001% to about 1% of at least one surfactant; about 1 mM to about 100 mM of a buffer; optionally about 10 mM to about 500 mM of a stabilizer; and about 5 mM to about 305 mM of a tonicity agent; and has a pH of about 4.0 to about 7.0.

As another example, a subject parenteral formulation is a liquid or lyophilized formulation comprising: about 1 mg/mL to about 200 mg/mL of a subject antibody; 0.04% Tween 20 w/v; 20 mM L-histidine; and 250 mM Sucrose; and has a pH of 5.5.

As another example, a subject parenteral formulation comprises a lyophilized formulation comprising: 1) 15 mg/mL of a subject antibody; 0.04% Tween 20 w/v; 20 mM L-histidine; and 250 mM sucrose; and has a pH of 5.5; or 2) 75 mg/mL of a subject antibody; 0.04% Tween 20 w/v; 20 mM L-histidine; and 250 mM sucrose; and has a pH of 5.5; or 3) 75 mg/mL of a subject antibody; 0.02% Tween 20 w/v; 20 mM L-histidine; and 250 mM Sucrose; and has a pH of 5.5; or 4) 75 mg/mL of a subject antibody; 0.04% Tween 20 w/v; 20 mM L-histidine; and 250 mM trehalose; and has a pH of 5.5; or 6) 75 mg/mL of a subject antibody; 0.02% Tween 20 w/v; 20 mM L-histidine; and 250 mM trehalose; and has a pH of 5.5.

As another example, a subject parenteral formulation is a liquid formulation comprising: 1) 7.5 mg/mL of a subject antibody; 0.022% Tween 20 w/v; 120 mM L-histidine; and 250 125 mM sucrose; and has a pH of 5.5; or 2) 37.5 mg/mL of a subject antibody; 0.02% Tween 20 w/v; 10 mM L-histidine; and 125 mM sucrose; and has a pH of 5.5; or 3) 37.5 mg/mL of a subject antibody; 0.01% Tween 20 w/v; 10 mM L-histidine; and 125 mM sucrose; and has a pH of 5.5; or 4) 37.5 mg/mL of a subject antibody; 0.02% Tween 20 w/v; 10 mM L-histidine; 125 mM trehalose; and has a pH of 5.5; or 5) 37.5 mg/mL of a subject antibody; 0.01% Tween 20 w/v; 10 mM L-histidine; and 125 mM trehalose; and has a pH of 5.5; or 6) 5 mg/mL of a subject antibody; 0.02% Tween 20 w/v; 20 mM L-histidine; and 250 mM trehalose; and has a of 5.5; or 7) 75 mg/mL of a subject antibody; 0.02% Tween 20 w/v; 20 mM L-histidine; and 250 mM mannitol; and has a pH of 5.5; or 8) 75 mg/mL of a subject antibody; 0.02% Tween 20 w/v; 20 mM L histidine; and 140 mM sodium chloride; and has a pH of 5.5; or 9) 150 mg/mL of a subject antibody; 0.02% Tween 20 w/v; 20 mM L-histidine; and 250 mM trehalose: and has a pH of 5.5; or 10) 150 mg/mL of a subject antibody; 0.02% Tween 20 w/v; 20 mM L-histidine; and 250 mM mannitol; and has a pH of 5.5; or 11) 150 mg/mL of a subject antibody; 0.02% Tween 20 w/v; 20 mM L-histidine; and 140 mM sodium chloride; and has a pH of 5.5; or 12) 10 mg/mL of a subject antibody; 0.01% Tween 20 w/v; 20 mM L-histidine; and 40 mM sodium chloride; and has a pH of 5.5.

A subject antibody can be utilized in aerosol formulation to be administered via inhalation. A subject antibody can be formulated into pressurized acceptable propellants such as dichlorodifluoromethane, propane, nitrogen and the like.

Furthermore, a subject antibody can be made into suppositories by mixing with a variety of bases such as emulsifying bases or water-soluble bases. A subject antibody can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the subject antibody (ies). Similarly, unit dosage forms for injection or intravenous administration may comprise a subject antibody in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of a subject Collagen I binding agent calculated in an amount sufficient to produce the desired effect in association with a pharmaceutically acceptable diluent, carrier or vehicle. The specifications for a subject Collagen I binding agent may depend on the particular Collagen I binding agent employed and the effect to be achieved, and the pharmacodynamics associated with each antibody in the host.

Other modes of administration will also find use in a subject method. For instance, a subject antibody can be formulated in suppositories and, in some cases, aerosol and intranasal compositions. For suppositories, the vehicle composition will include traditional binders and carriers such as, polyalkylene glycols, or triglycerides. Such suppositories may be formed from mixtures containing the active ingredient in the range of about 0.5% to about 10% (w/w), e.g., about 1% to about 2%.

Intranasal formulations will usually include vehicles that neither cause irritation to the nasal mucosa nor significantly disturb ciliary function. Diluents such as water, aqueous saline or other known substances can be employed. The nasal formulations may also contain preservatives such as, but not limited to, chlorobutanol and benzalkonium chloride. A surfactant may be present to enhance absorption of the subject proteins by the nasal mucosa.

A subject antibody can be administered as an injectable formulation. Typically, injectable compositions are prepared as liquid solutions or suspensions; solid forms suitable for solution in, or suspension in, liquid vehicles prior to injection may also be prepared. The preparation may also be emulsified or the antibody encapsulated in liposome vehicles.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of a subject antibody adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, a subject antibody is formulated in a controlled release formulation. Sustained-release preparations may be prepared using methods well known in the art. Suitable examples of sustained-release preparations include semipermeable matrices of solid hydrophobic polymers containing the antibody in which the matrices are in the form of shaped articles, e.g. films or microcapsules. Examples of sustained-release matrices include polyesters, copolymers of L-glutamic acid and ethyl-L-glutamate, non-degradable ethylene-vinyl acetate, hydrogels, polylactides, degradable lactic acid-glycolic acid copolymers and poly-D-(−)-3-hydroxybutyric acid. Possible loss of biological activity and possible changes in immunogenicity of antibodies comprised in sustained-release preparations may be prevented by using appropriate additives, by controlling moisture content and by developing specific polymer matrix compositions.

Controlled release can be taken to mean any one of a number of extended release dosage forms. The following terms may be considered to be substantially equivalent to controlled release: continuous release, controlled release, delayed release, depot, gradual release, long-term release, programmed release, prolonged release, proportionate release, protracted release, repository, retard, slow release, spaced release, sustained release, time coat, timed release, delayed action, extended action, layered-time action, long acting, prolonged action, repeated action, slowing acting, sustained action, sustained-action medications, and extended release. Further discussions of these terms may be found in Lesczek Krowczynski, Extended-Release Dosage Forms, 1987 (CRC Press, Inc.).

The various controlled release technologies cover a very broad spectrum of drug dosage forms. Controlled release technologies include, but are not limited to physical systems and chemical systems.

Physical systems include, but are not limited to, reservoir systems with rate-controlling membranes, such as microencapsulation, macroencapsulation, and membrane systems; reservoir systems without rate-controlling membranes, such as hollow fibers, ultra-microporous cellulose triacetate, and porous polymeric substrates and foams; monolithic systems, including those systems physically dissolved in non-porous, polymeric, or elastomeric matrices (e.g., nonerodible, erodible, environmental agent ingression, and degradable), and materials physically dispersed in non-porous, polymeric, or elastomeric matrices (e.g., nonerodible, erodible, environmental agent ingression, and degradable); laminated structures, including reservoir layers chemically similar or dissimilar to outer control layers; and other physical methods, such as osmotic pumps, or adsorption onto ion-exchange resins.

Chemical systems include, but are not limited to, chemical erosion of polymer matrices (e.g., heterogeneous, or homogeneous erosion), or biological erosion of a polymer matrix (e.g., heterogeneous, or homogeneous). Additional discussion of categories of systems for controlled release may be found in Agis F. Kydonieus, Controlled Release Technologies: Methods, Theory and Applications, 1980 (CRC Press, Inc.).

There are a number of controlled release drug formulations that are developed for oral administration. These include, but are not limited to, osmotic pressure-controlled gastrointestinal delivery systems; hydrodynamic pressure-controlled gastrointestinal delivery systems; membrane permeation-controlled gastrointestinal delivery systems, which include microporous membrane permeation-controlled gastrointestinal delivery devices; gastric fluid-resistant intestine targeted controlled-release gastrointestinal delivery devices; gel diffusion-controlled gastrointestinal delivery systems; and ion-exchange-controlled gastrointestinal delivery systems, which include cationic and anionic drugs. Additional information regarding controlled release drug delivery systems may be found in Yie W. Chien, Novel Drug Delivery Systems, 1992 (Marcel Dekker, Inc.).

A suitable dosage can be determined by an attending physician or other qualified medical personnel, based on various clinical factors. As is well known in the medical arts, dosages for any one patient depend upon many factors, including the patient's size, body surface area, age, the particular compound to be administered, sex of the patient, time, and route of administration, general health, and other drugs being administered concurrently. A subject antibody may be administered in amounts between 1 ng/kg body weight and 20 mg/kg body weight per dose, e.g. between 0.1 mg/kg body weight to 10 mg/kg body weight, e.g. between 0.5 mg/kg body weight to 5 mg/kg body weight; however, doses below or above this exemplary range are envisioned, especially considering the aforementioned factors. If the regimen is a continuous infusion, it can also be in the range of 1 μg to 10 mg per kilogram of body weight per minute.

Those of skill will readily appreciate that dose levels can vary as a function of the specific antibody, the severity of the symptoms and the susceptibility of the subject to side effects. Preferred dosages for a given compound are readily determinable by those of skill in the art by a variety of means.

Routes of Administration

A subject antibody is administered to an individual using any available method and route suitable for drug delivery, including in vivo and ex vivo methods, as well as systemic and localized routes of administration.

Conventional and pharmaceutically acceptable routes of administration include intranasal, intramuscular, intratracheal, subcutaneous, intradermal, topical application, intravenous, intraarterial, rectal, nasal, oral, and other enteral and parenteral routes of administration. Routes of administration may be combined, if desired, or adjusted depending upon the antibody and/or the desired effect. A subject antibody composition can be administered in a single dose or in multiple doses. In some embodiments, a subject antibody composition is administered orally. In some embodiments, a subject antibody composition is administered via an inhalational route. In some embodiments, a subject antibody composition is administered intranasally. In some embodiments, a subject antibody composition is administered locally. In some embodiments, a subject antibody composition is administered intracranially. In some embodiments, a subject antibody composition is administered intravenously.

The agent can be administered to a host using any available conventional methods and routes suitable for delivery of conventional drugs, including systemic or localized routes. In general, routes of administration contemplated for use include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrasternal, and intravenous routes, i.e., any route of administration other than through the alimentary canal. Parenteral administration can be carried to effect systemic or local delivery of a subject antibody. Where systemic delivery is desired, administration typically involves invasive or systemically absorbed topical or mucosal administration of pharmaceutical preparations.

A subject antibody can also be delivered to the subject by enteral administration. Enteral routes of administration include, but are not necessarily limited to, oral and rectal (e.g., using a suppository) delivery.

By “treatment” is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as cancer, and pain associated therewith. As such, treatment also includes situations in which the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

In some embodiments, a subject antibody is administered by injection and/or delivery, e.g., to a site in a brain artery or directly into brain tissue. A subject antibody can also be administered directly to a target site e.g., by biolistic delivery to the target site.

A variety of hosts (wherein the term “host” is used interchangeably herein with the terms “subject,” “individual,” and “patient”) are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans: and non-human primates such as chimpanzees and monkeys). In some embodiments, the hosts will be humans.

### REFERENCES

1. Chung H J, Steplewski A, Chung K Y, Uitto J, Fertala A: Collagen fibril formation. A new target to limit fibrosis, J Biol Chem 2008, 283:25879-25886
2. Fertala J, Steplewski A, Kostas J, Beredjiklian P, Williams G, Arnold W, Abboud J, Bhardwaj A, Hou C, Fertala A: Engineering and characterization of the chimeric antibody that targets the C-terminal telopeptide of the alpha2 chain of human collagen I: a next step in the quest to reduce localized fibrosis, Connect Tissue Res 2013, 54:187-196
3. Fertala J, Kostas J, Hou C, Steplewski A, Beredjiklian P, Abboud J, Arnold W V, Williams G, Fertala A: Testing the anti-fibrotic potential of the single-chain Fv antibody against the alpha2 C-terminal telopeptide of collagen I, Connect Tissue Res 2014, 55:115-122
4. Rivlin M, Arnold W V, Kostas J, Hou C, Fertala A: Testing the Utility of Engineered Anti-Collagen I Antibody to Limit the Formation of Collagen-Rich Fibrotic Deposits in a Rabbit Model of Posttraumatic Joint Stiffness. Edited by 2015, p.
5. Prockop D J, Fertala A: Inhibition of the self-assembly of collagen I into fibrils with synthetic peptides. Demonstration that assembly is driven by specific binding sites on the monomers, J Biol Chem 1998, 273:15598-15604
6. Steplewski A, Fertala A: Inhibition of collagen fibril formation, Fibrogenesis Tissue Repair 2012, 5 Suppl 1:S29
7. Steplewski A, Fertala J, Beredjiklian P, Wang M L, Fertala A: Matrix-specific anchors: a new concept for targeted delivery and retention of therapeutic cells, Tissue engineering Part A 2015, 21:1207-1216
8. Steplewski A, Fertala J, Beredjiklian P K, Abboud J A, Wang M L, Namdari S, Barlow J, Rivlin M, Arnold W V, Kostas J, Hou C, Fertala A: Testing the Utility of Engineered Anti-Collagen I Antibody to Limit the Formation of Collagen-Rich Fibrotic Deposits in a Rabbit Model of Posttraumatic Joint Stiffness. Edited by 2015, p.
9. Wynn TA: Cellular and molecular mechanisms of fibrosis, The Journal of pathology 2008, 214:199-210

1. A monoclonal antibody comprising the amino acid sequences of the complementarity determining regions (CDRs) of the heavy alpha chain corresponding to and the light kappa chain corresponding to of a monoclonal antibody (denoted as anti-fibrotic antibody, AFA) that blocks the binding activity of the C-terminal telopeptide region of human collagen I (denoted as CTTR1) consisting of two α1(I)C-telopeptides (denoted as α1Ct) and one α2(I)C-telopeptide (denoted as α2Ct).

2. The monoclonal antibody of claim 1 wherein the CDRs mediate the blocking of the CTTR1 via binding to its specific subdomain.

3. The monoclonal antibody of claim 1 wherein the CDRs mediate the binding interaction with a specific epitope, (denoted as A2_DGDFY) present within the α2Ct, with a minimum binding affinity of 22 μM.

4. The monoclonal antibody of claim 1 having the sequence according to SEQ ID No 2 for the heavy alpha chain.

5. The monoclonal antibody of claim 1 comprising CDR's having the sequences according to SEQ ID Nos 3, 4, and 5 for the heavy alpha chain.

6. The monoclonal antibody of claim 1 having the sequence according to SEQ ID No 6 for the light kappa chain.

7. The monoclonal antibody of claim 1 comprising CDR's having the sequence according to SEQ ID Nos 7, 8, and 9 for the light kappa chain.

8. A monoclonal antibody-based biologics in systemic or localized fibrotic diseases to limit the progression of the fibrotic process.

9. The monoclonal antibody of claim 8 having a heavy alpha chain and a light kappa chain.

10. The monoclonal antibody of claim 9 wherein the heavy alpha chain corresponds to SEQ ID NO 2.

11. The monoclonal antibody of claim 9 wherein the heavy alpha chain comprises SEQ ID Nos 3, 4, and 5.

12. The monoclonal antibody of claim 9 wherein the light kappa chain corresponds to SEQ ID NO. 6.

13. The monoclonal antibody of claim 9 wherein the light kappa chain comprises SEQ ID Nos 7, 8, and 9.

14. The monoclonal antibody of claim 8, wherein the secondary use of this invention includes targeted delivery of therapeutic compounds to collagen I-rich connective tissues.

15. The monoclonal antibody of claim 8 wherein the antibody has a highly-specific binding mediated by the described CDRs-CTTR1 interaction may serve to deliver therapeutic agents including antibiotics, growth factors, therapeutic cells, and others.

16. An anti-fibrotic biologic comprising, a full-length chimeric IgG variant, a humanized IgG variant, a scFv variant, or other active biologic including the entire CDRs or their fragments able to bind to the α2Ct target.

17. The anti-fibrotic biologic of claim 16 comprising a heavy chain corresponding to SEQ ID No. 2.

18. The anti-fibrotic biologic of claim 16 comprising a light chain corresponding to SEQ ID No. 6.

19. The anti-fibrotic biologic of claim 16 wherein the CDR of the heavy chain comprises SEQ ID Nos. 3, 4, and 5.

20. The anti-fibrotic biologic of claim 16 wherein the CDR of the light chain comprises SEQ ID Nos. 7, 8, and 9.

21. The anti-fibrotic biologic of claim 16 further comprising a homology to SEQ ID No. 2 of at least 90%.

22. The anti-fibrotic biologic of claim 16 further comprising a homology to SEQ ID No. 6 of at least 90%.

23. The anti-fibrotic biologic of claim 16 wherein said anti-fibrotic biologic comprises a further component selected from the group consisting of: a linked polymer, glycosylated, radiolabeled, covalently linked to a moiety, immobilized on a solid support, linked to a toxin, a chemotherapeutic, or an imaging compound; or combinations thereof.

24. The monoclonal antibody of claim 1, wherein said antibody comprises a further component selected from the group consisting of: a linked polymer, glycosylated, radiolabeled, covalently linked to a moiety, immobilized on a solid support, linked to a toxin, a chemotherapeutic, or an imaging compound; or combinations thereof.

25. A pharmaceutical composition comprising an antibody having a variable chain of SEQ ID No. 2, and of SEQ ID No. 6.

26. A method of treating excessive fibrotic tissue formation in a patient comprising administering to said patient an effective amount of the pharmaceutical composition of claim 25.

27. A pharmaceutical composition comprising an antibody having CDR's corresponding to SEQ ID Nos. 3, 4, 5, in the heavy chain and 7, 8, and 9 in the light chain.

28. A method of treating excessive fibrotic tissue formation in a patient comprising administering to said patient an effective amount of the pharmaceutical composition of claim 27.

29. A method of limiting growth of fibrotic tissue by blocking collagen fibril formation comprising administering to a patient an effective amount of an anti-fibrotic antibody.

30. The method of claim 29 wherein the anti-fibrotic antibody comprises a sequence comprising SEQ ID No. 2 and SEQ ID No. 6.

31. The method of claim 29 wherein the anti-fibrotic antibody comprises CDR's in a light and heavy chain, comprising SEQ ID Nos. 3, 4, and 5, in the heavy chain and SEQ ID Nos. 7, 8, and 9 in the light chain.

32. A method of delivering targeted therapeutic compounds to collagen I rich connective tissues comprising administering to a patient an effective amount of an antibody having affinity for collagen I rich tissues, and comprising a therapeutic compound bound to said antibody.

33. The method of claim 32 wherein the anti-fibrotic antibody comprises a sequence comprising SEQ ID No. 2 and SEQ ID No. 6.

34. The method of claim 32 wherein the anti-fibrotic antibody comprises CDR's in a light and heavy chain, comprising SEQ ID Nos. 3, 4, and 5, in the heavy chain and SEQ ID Nos. 7, 8, and 9 in the light chain.

35. The method of claim 32 wherein the therapeutic compound is selected from the group consisting of an antibiotic, a growth factor, therapeutic cells, and a chemotherapeutic agent.

36. The method of claim 32, wherein the therapeutic compound is administered via systemic delivery, local delivery via injection at a wound site, or topical application in the form of an ointment, drops, or spray.
